(19) United States
(12) Patent Application Publication
(10) Pub. No.: US 2026/0298810 A1
(43) Pub. Date: Oct. 1, 2026

(54) SPECTROMETER DEVICE FOR OBTAINING SPECTROSCOPIC INFORMATION ON AT LEAST ONE OBJECT

(71) Applicant: TRINAMIX GMBH, Ludwigshafen am Rhein (DE)

(72) Inventors: Henning ZIMMERMANN, Ludwigshafen am Rhein (DE); Felix SCHMIDT, Ludwigshafen am Rhein (DE); Szu-Yu HUANG, Ludwigshafen am Rhein (DE); Christoph PROKOP, Ludwigshafen am Rhein (DE); Celal Mohan OEGUEN, Ludwigshafen am Rhein (DE)

(21) Appl. No.: 19/474,527

(22) PCT Filed: Apr. 23, 2024
(86) PCT No.: PCT/EP24/61050

§ 371 Date: Oct. 10, 2025

(30) Foreign Application Priority Data
Apr. 24, 2023 (EP) 23169494.4

(51) Int. Cl. G01N 21/31; A61B 5/00

(52) U.S. Cl. CPC G01N 21/31; A61B 5/0075; G01N 2201/0636; G01N 2201/127

(57) ABSTRACT

Disclosed herein is a spectrometer device for obtaining spectroscopic information on at least one object. The spectrometer device includes:
at least one detector for detecting light from an object;
at least one optical filter configured for transferring incident light within at least one selected wavelength range onto the detector;
at least one light source configured for emitting light in at least one optical spectral range;
at least one sample interface configured for allowing the emitted light of the light source to illuminate an object held by the sample interface and configured for allowing light from the object held by the sample interface to propagate via the optical filter to the detector;
at least one first optical path; and
at least one second optical path.

### TECHNICAL FIELD

The invention relates to spectrometer device and several uses. Such device can, in general, be used for investigating or monitoring purposes, in particular, in the infrared (IR) spectral region, especially in the near-infrared (NIR) spectral region, and in the visible (VIS) spectral region. However, further applications are feasible.

### BACKGROUND ART

In general, spectrometers are known to collect information on the spectral light composition from an object, when irradiating, reflecting and/or absorbing light. In order to allow comparing spectra from multiple spectrometers, the spectrometers have to be calibrated. In general, spectrometers require a wavelength or wavenumber calibration, e.g. a calibration of the x-axis of a recorded spectrum, and a calibration of the signal, reflectance, transmittance and/or absorbance, i.e. a calibration of the y-axis of the recorded spectrum. For example, calibrations of the y-axis of the recorded spectrum for reflective measurements, which is generally required in the field of diffusive reflective near-infrared spectroscopy, may use external reflection standards which are placed at a object position. Alternatively, the calibration may use internal calibration targets which are automatically moved into a measurement field by the spectrometer itself to calibrate the spectrometer's response.

However, in the field of mobile spectroscopy, the use of external calibration standards and/or moving internal calibration targets may be not feasible. Thus, calibration schemes are known which avoid the need for placing external calibration standards at the object position and/or moving internal calibration targets. These calibration schemes may use internal calibration paths.

US 2021/0063241 A1 describes an optical measurement device including a light source; an emission optic configured to direct a first portion of light generated by the light source to a measurement target; a collection optic configured to receive light from the measurement target; an optical conduit configured to direct a second portion of light generated by the light source to a spectral reference; the spectral reference; a sensor; and a filter. A first portion of the filter may be provided between the collection optic and a first portion of the sensor. A second portion of the filter may be provided between the spectral reference and a second portion of the sensor.

US 2021/0356322 A1 describes a method of calibrating an optical sensor including acquiring a first characteristic for an external light source through a detector of an optical sensor while an internal light source of the optical sensor is turned off; driving the internal light source; acquiring a second characteristic for the internal light source and the external light source through the detector, based on driving the internal light source; and acquiring a reference characteristic of the internal light source, for calculation of an absorbance of an object, based on the first characteristic and the second characteristic.

Despite the advantages achieved by known methods and devices, several technical challenges remain. Generally, calibrations of the spectrometer aim in a constant cᵢ=S_{1,i}/S_{2,i}=const for all temperatures, wherein cᵢ refers to a calibration factor of the filters with wavelength λᵢ. If the calibration constant cᵢ is not constant, the calibration of the spectrometer usually fails. For calibrations schemes using internal calibration paths, the calibration factor may be determined using signals S₁ and S₂ which depend on a spectral flux of a light source Φ_{Light} being generally temperature-dependent. Further, the signals may depend on a reflectance of the external reflection target on an external path Rₑₓₜ, a reflectance of the internal reflection target in an internal path Rᵢₙₜ, and a transmissivity of optical filters in the external path T_{2,filter} and the internal path T_{1,filter}:

$${S}_{1 , i} = \int {Φ}_{Light} ( λ , Temp ) \cdot {R}_{i n t} ( λ ) \cdot {T}_{1 , filter} ( λ , {λ}_{i} ) d λ \\ {S}_{2 , i} = \int {Φ}_{Light} ( λ , Temp ) \cdot {R}_{ext} ( λ ) \cdot {T}_{2 , filter} ( λ , {λ}_{i} ) d λ$$

Accordingly, the spectral flux of the light source Φ_{Light} is weighted differently when the spectral flux changes with temperature. This different weighting may result in a change of the calibration factor c, if either Rₑₓₜ≠Rᵢₙₜ and/or T_{1,filter}≠T_{2,filter}. Thus, when using a temperature-dependent light source for the spectrometer in combination with an angle-of-incidence (AOI) dependent optical filter, a change in temperature may lead to a drift of the calibration factor of the spectrometer. The drift of the calibration factor may specifically comprise a change over temperature in the ratio between light in the internal path and the external path. Consequently, the drift of the calibration factor may cause a temperature-induced drift of the recorded spectrum. This may be especially critical if the temperature dependence of the light source is varying over its emission spectrum.

Moreover, on the one hand miniature spectroscopy requires low footprint and a compact design. On the other hand high performance spectroscopy requires blocking of straylight to achieve high dynamic range of the spectrometer. No light should evade the module out of the predefined optical path such that materials need to be opaque.

### Problem to be Solved

It is therefore desirable to provide a spectrometer device which at least partially address above-mentioned technical challenges of known devices. Specifically, a spectrometer device shall be provided which allow a temperature-robust internal calibration for miniaturized NIR spectroscopy.

### SUMMARY

This problem is addressed by a spectrometer device with the features of the independent claim. Advantageous embodiments which might be realized in an isolated fashion or in any arbitrary combinations are listed in the dependent claims as well as throughout the specification.

In a first aspect of the present invention, a spectrometer device for obtaining spectroscopic information on at least one object is disclosed.

The spectrometer device comprises
at least one detector for detecting light from an object;
at least one optical filter configured for transferring incident light within at least one selected wavelength range onto the detector;
at least one light source configured for emitting light in at least one optical spectral range;
at least one sample interface configured for allowing the emitted light of the light source to illuminate an object held by the sample interface and configured for allowing light from the object held by the sample interface to propagate via the optical filter to the detector, wherein the sample interface comprises at least one module cover window comprising at least one substrate, wherein the module cover window comprises at least one internal calibration target partially covering the substrate;
at least one first optical path, wherein the first optical path is configured for allowing the emitted light of the light source to propagate via the optical filter to the detector by passing the sample interface at least once; and
at least one second optical path, wherein the second optical path is configured for allowing the emitted light of the light source to propagate via the optical filter to the detector by interacting with the internal calibration target at least once.

The term “spectrometer device” as used herein is a broad term and is to be given its ordinary and customary meaning to a person of ordinary skill in the art and is not to be limited to a special or customized meaning. The term specifically may refer, without limitation, to an optical device configured for acquiring at least one item of spectral information on at least one object.

Specifically, the at least one item of spectral information may refer to at least one optical property or optically measurable property which is determined as a function of a wavelength, for one or more different wavelengths. More specifically, the optical property or optically measurable property, as well as the at least one item of spectral information, may relate to at least one property characterizing at least one of a transmission, an absorption, a reflection and an emission of the at least one object, either by itself or after illumination with external light. The at least one optical property may be determined for one or more wavelengths. The spectrometer device specifically may form an apparatus which is capable of recording a signal intensity with respect to the corresponding wavelength of a spectrum or a partition thereof, such as a wavelength interval, wherein the signal intensity may, specifically, be provided as an electrical signal which may be used for further evaluation.

The spectrometer device, as an example, may be or may comprise a device which allows for a measurement of at least one spectrum, e.g. for the measurement of a spectral flux, specifically as a function of a wavelength or detection wavelength. The spectrum may be acquired, as an example, in absolute units or in relative units, e.g. in relation to at least one reference measurement. Thus, as an example, the acquisition of the at least one spectrum specifically may be performed either for a measurement of the spectral flux (unit W/nm) or for a measurement of a spectrum relative to at least one reference material (unit 1), which may describe the property of a material, e.g., reflectance over wavelength. Additionally or alternatively, the reference measurement may be based on a reference light source, an optical reference path, a calculated reference signal, e.g. a calculated reference signal from literature, and/or on a reference device.

The spectrometer device may be a diffusive reflective spectrometer device configured for acquiring spectral information from the light which is diffusively reflected by the at least one object, e.g. at least one object. Additionally or alternatively, the at least one spectrometer device may be or may comprise an absorption- and/or transmission spectrometer. In particular, measuring a spectrum with the spectrometer device may comprise measuring reflectance in a reflective configuration. Specifically, the spectrometer device may be configured for measuring reflectance in a reflective configuration. As outlined above, however, other types of spectrometer devices are also feasible.

The spectrometer device may comprise at least one light source which, as an example, may be at least one of a tunable light source, a light source having at least one fixed emission wavelength and a broadband light source. The spectrometer device, as will be outlined in further detail below, further comprises at least one detector configured for detecting electromagnetic radiation, in particular light, such as light which is at least one of transmitted, reflected or emitted from the at least one object. The spectrometer device further may comprise, as will be outlined in further detail below, at least one wavelength-selective element, such as at least one of a grating, a prism and a filter, e.g. a length variable filter having varying transmission properties over its lateral extension. The wavelength-selective element may be used for separating incident light into a spectrum of constituent wavelength signals whose respective intensities are determined by employing a detector such as a detector having a detector array as described below in more detail.

The spectrometer device may specifically be a portable spectrometer device. For example, the portable spectrometer device may be part of a mobile device or may be attachable to a mobile device, such as a notebook computer, a tablet, a cell phone, such as a smart phone, a smartwatch and/or a wearable computer or the like.

The term “spectroscopic information”, also referred to as “spectral information” or as “an item of spectral information”, as used herein is a broad term and is to be given its ordinary and customary meaning to a person of ordinary skill in the art and is not to be limited to a special or customized meaning. The term specifically may refer, without limitation, to an item of information, e.g. on at least one object and/or radiation emitted by at least one object, characterizing at least one optical property of the object, more specifically at least one item of information characterizing, e.g. qualifying and/or quantifying, at least one of a transmission, an absorption, a reflection and an emission of the at least one object. As an example, the at least one item of spectral information may comprise at least one intensity information, e.g. information on an intensity of light being at least one of transmitted, absorbed, reflected or emitted by the object, e.g. as a function of a wavelength or wavelength sub-range over one or more wavelengths, e.g. over a range of wavelengths. Specifically, the intensity information may correspond to or be derived from the signal intensity, specifically the electrical signal, recorded by the spectrometer device with respect to a wavelength or a range of wavelengths of the spectrum.

The spectrometer device specifically may be configured for obtaining at least one spectrum or at least a part of a spectrum of detection light propagating from the object to the spectrometer device. The spectrum may describe the radiometric unit of spectral flux, e.g. given in units of watt per nanometer (W/nm), or other units, e.g. as a function of the wavelength of the detection light. Thus, the spectrum may describe the optical power of light, e.g. in the NIR spectral range, in a specific wavelength band. The spectrum may contain one or more optical variables as a function of the wavelength, e.g. the power spectral density, electric signals derived by optical measurements and the like. The spectrum may indicate, as an example, the power spectral density and/or the spectral flux of the object, e.g. of a object, e.g. relative to a reference object, such as a transmittance and/or a reflectance of the object, specifically of the object.

The spectrometer device may be configured for obtaining the spectrum in wavelength range at least partially comprising one or more of an infrared, a visible and an ultraviolet spectral range.

The spectrometer device may be a near-infrared spectrometer. For example, the spectrometer device may be configured for obtaining the spectrum in a wavelength range at least partially comprising the near-infrared spectral range, such as in a wavelength range from 760 nm to 5 μm, specifically in a wavelength range from 1 μm to 3 μm.

The spectrum, as an example, may comprise at least one measurable optical variable or property of the detection light and/or of the object, specifically as a function of illumination light and/or detection light. As an example, the at least one measurable optical variable or property may comprise at least one at least one radiometric quantity, such as at least one of a spectral density, a power spectral density, a spectral flux, a radiant flux, a radiant intensity, a spectral radiant intensity, an irradiance, a spectral irradiance. Specifically, as an example, the spectrometer device, specifically the detector, may measure the irradiance in Watt per square meter (W/m²), more specifically the spectral irradiance in Watt per square meter per nanometer (W/m²/nm). Based on the measured quantity the spectral flux in Watt per nanometer (W/nm) and/or the radiant flux in Watt (W) may be determined, e.g. calculated, by taking into account an area of the detector.

The term “object” as used herein is a broad term and is to be given its ordinary and customary meaning to a person of ordinary skill in the art and is not to be limited to a special or customized meaning. The term specifically may refer, without limitation, to an arbitrary body, chosen from a living object and a non-living object. Thus, as an example, the at least one object may comprise one or more articles and/or one or more parts of an article, wherein the at least one article or the at least one part thereof may comprise at least one component which may provide a spectrum suitable for investigations. Additionally or alternatively, the object may be or may comprise one or more living beings and/or one or more parts thereof, such as one or more body parts of a human being, e.g. a user, and/or an animal. The object specifically may comprise at least one sample which may fully or partially be analyzed by spectroscopic methods. As an example, the object may be or may comprise at least one of: human or animal skin; edibles, such as fruits; plastics and textile.

The spectrometer device comprises at least one detector for detecting light, in particular detection light, from the object. The term “to detect” as used herein is a broad term and is to be given its ordinary and customary meaning to a person of ordinary skill in the art and is not to be limited to a special or customized meaning. The term specifically may refer, without limitation, to the process of at least one of determining, measuring and monitoring at least one parameter, qualitatively and/or quantitatively, such as at least one of a physical parameter, a chemical parameter and a biological parameter. Specifically, the physical parameter may be or may comprise an electrical parameter. Consequently, the term “detector” as used herein is a broad term and is to be given its ordinary and customary meaning to a person of ordinary skill in the art and is not to be limited to a special or customized meaning. The term specifically may refer, without limitation, to an arbitrary device configured for detecting, i.e. for at least one of determining, measuring and monitoring, at least one parameter, qualitatively and/or quantitatively, such as at least one of a physical parameter, a chemical parameter and a biological parameter. The detector may be configured for generating at least one detector signal, more specifically at least one electrical detector signal, such as an analogue and/or a digital detector signal, the detector signal providing information on the at least one parameter measured by the detector. The detector signal may directly or indirectly be provided by the detector to at least one evaluation unit, such that the detector and the evaluation unit may be directly or indirectly connected. The detector signal may be used as a “raw” detector signal and/or may be processed or preprocessed before further used, e.g. by filtering and the like. Thus, the detector may comprise at least one processing device and/or at least one preprocessing device, such as at least one of an amplifier, an analogue/digital converter and an electrical filter.

In the present case, the detector is configured for detecting light propagating from the object to the spectrometer device or more specifically to the detector of the spectrometer device, which is also referred to as “detection light”. Thus, specifically, the detector may be or may comprise at least one optical detector. The optical detector may be configured for determining at least one optical parameter, such as an intensity and/or a power of light by which at least one sensitive area of the detector is irradiated. More specifically, the optical detector may comprise at least one photosensitive element and/or at least one optical sensor, such as at least one of a photodiode, a photocell, a photosensitive resistor, a phototransistor, a thermophile sensor, a photoacoustic sensor, a pyroelectric sensor, a photomultiplier and a bolometer. The detector, thus, may be configured for generating at least one detector signal, more specifically at least one electrical detector signal, in the above-mentioned sense, providing information on at least one optical parameter, such as the power and/or intensity of light by which the detector or a sensitive area of the detector is illuminated.

The detector may comprise one single optically sensitive element or area or a plurality of optically sensitive elements or areas. Specifically, the detector may be or may comprise at least one detector array, more specifically an array of photosensitive elements. Each of the photosensitive elements may comprise at least a photosensitive area which may be adapted for generating an electrical signal depending on the intensity of the incident light, wherein the electrical signal may, in particular, be provided to the evaluation unit, as will be outlined in further detail below.

The photosensitive area as comprised by each of the optically sensitive elements may, especially, be a single, uniform photosensitive area which is configured for receiving the incident light which impinges on the individual optically sensitive elements. However, other arrangements of the optically sensitive elements may also be conceivable.

The array of optically sensitive elements may be designed to generate detector signals, preferably electronic signals, associated with the intensity of the incident light which impinges on the individual optically sensitive elements. The detector signal may be an analogue and/or a digital signal. The electronic signals for adjacent pixelated sensors can, accordingly, be generated simultaneously or else in a temporally successive manner. By way of example, during a row scan or line scan, it is possible to generate a sequence of electronic signals which correspond to the series of the individual optically sensitive elements which are arranged in a line. In addition, the individual optically sensitive elements may, preferably, be active pixel sensors which may be adapted to amplify the electronic signals prior to providing it to the evaluation unit. For this purpose, the detector may comprise one or more signal processing devices, such as one or more filters and/or analogue-digital-converters for processing and/or preprocessing the electronic signals.

In case the detector comprises an array of optically sensitive elements, the detector, as an example, may be selected from any known pixel sensor, in particular, from a pixelated organic camera element, preferably, a pixelated organic camera chip, or from a pixelated inorganic camera element, preferably, a pixelated inorganic camera chip, more preferably from a CCD chip or a CMOS chip, which are, commonly, used in various cameras nowadays. As an alternative, the detector generally may be or comprise a photoconductor, in particular an inorganic photoconductor, especially PbS, PbSe, Ge, InGaAs, ext. InGaAs, InSb, or HgCdTe. As a further alternative it may comprise at least one of pyroelectric, bolometer or thermophile detector elements. Thus, a camera chip having a matrix of 1×N pixels or of M×N pixels may be used here, wherein, as an example, M may be <10 and N may be in the range from 1 to 50, preferably from 2 to 20, more preferred from 5 to 10. Further, a monochrome camera element, preferably a monochrome camera chip, may be used, wherein the monochrome camera element may be differently selected for each optically sensitive element, especially, in accordance with the varying wavelength along the series of the optical sensors. The array may be adapted to provide a plurality of the electrical signals which may be generated by the photosensitive areas of the optically sensitive elements comprised by the array. The electrical signals as provided by the array of the spectrometer device may be forwarded to the evaluation unit.

The spectrometer device further comprises at least one optical filter configured for transferring incident light within at least one selected wavelength range onto the detector. The term “optical filter” as used herein is a broad term and is to be given its ordinary and customary meaning to a person of ordinary skill in the art and is not to be limited to a special or customized meaning. The term specifically may refer, without limitation, to an arbitrary optical element configured for selectively transmitting light having different wavelengths. Specifically, the optical filter may be configured for transmitting light having a wavelength within the at least one selected wavelength range, wherein the transmitting of light having a wavelength outside the selected wavelength range may be at least partially prevented, such as by having a diminished transmissivity outside the selected wavelength range compared to the transmissivity in the selected wavelength range. Consequently, the term “selected wavelength range”, as used herein, is a broad term and is to be given its ordinary and customary meaning to a person of ordinary skill in the art and is not to be limited to a special or customized meaning and specifically may refer, without limitation, to at least one wavelength range of the optical filter allowing transmitting of light. For example, light having a wavelength with the selected wavelength range may be able to pass through the optical filter, wherein light having a wavelength outside the selected wavelength range may be at least partially blocked, such as by having a reduced light intensity after the optical filter. The optical filter may comprise at least one filter selected from the group consisting of: an interferometric filter; an absorption filter; a dichroic filter; a MEMS Fabry-Pérot interferometer. Specifically, the optical filter may comprise at least one narrow band pass filter, more specifically a set of narrow band pass filter. The narrow band pass filter may be configured for transmitting light only within a narrow selected wavelength range, such as a wavelength range having a width in the range of 10 to 100 nm, specifically in the range of 10 to 50 nm, more specifically a wavelength range having a width of 20 nm, most specifically a wavelength range having a width of 15 nm. For example, in the set of narrow band pass filter, each of the narrow band pass filter may have a narrow selected wavelength range at least partially differing from each other.

A transmission profile of the optical filter may be dependent on an angle-of-incidence. The term “transmission profile” as used herein is a broad term and is to be given its ordinary and customary meaning to a person of ordinary skill in the art and is not to be limited to a special or customized meaning. The term specifically may refer, without limitation, to transmission as a function of wavelength. Specifically, the transmission profile may indicate the optical filter's ability to transmit light as a function of wavelength. The transmission profile may comprise a numerical indication of the optical filter's ability to transmit light as a function of wavelength. For example, the transmission profile may comprise, for each wavelength in the selected wavelength range, a numerical indication quantifying a portion of transmitted light through the optical filter. The transmission profile may specifically comprise a ratio quantifying an amount of light transmitted by the optical filter relative to an amount of incident light. For example, the transmission profile may comprise a ratio quantifying a power of light transmitted by the optical filter compared with a power of incident light. The transmission profile may specifically be wavelength dependent, such as by having different ratios of transmitted light for different wavelengths of incident light. The term “angle-of-incidence” as used herein is a broad term and is to be given its ordinary and customary meaning to a person of ordinary skill in the art and is not to be limited to a special or customized meaning. The term specifically may refer, without limitation, to an angle under which incident light impinges onto a surface normal of a surface of an optical element. The surface normal of a surface may refer to a direction being perpendicular to the specific surface. The angle-of-incidence may specifically be an angle of incident light to a surface normal of a surface of the optical filter.

The spectrometer device further comprises at least one light source configured for emitting light, also denoted as illumination light herein, in at least one optical spectral range. The term “light” as used herein is a broad term and is to be given its ordinary and customary meaning to a person of ordinary skill in the art and is not to be limited to a special or customized meaning. The term specifically may refer, without limitation, to electromagnetic radiation in one or more of the infrared, the visible and the ultraviolet spectral range. Herein, the term “ultraviolet spectral range”, generally, refers to electromagnetic radiation having a wavelength of 1 nm to 380 nm, preferably of 100 nm to 380 nm. Further, in partial accordance with standard ISO-21348 in a valid version at the date of this document, the term “visible spectral range”, generally, refers to a spectral range of 380 nm to 760 nm. The term “infrared spectral range” (IR) generally refers to electromagnetic radiation of 760 nm to 1000 μm, wherein the range of 760 nm to 1.5 μm is usually denominated as “near infrared spectral range” (NIR) while the range from 1.5μ to 15 μm is denoted as “mid infrared spectral range” (MidIR) and the range from 15 μm to 1000 μm as “far infrared spectral range” (FIR). Preferably, light used for the typical purposes of the present invention is light in the infrared (IR) spectral range, more preferred, in the near infrared (NIR) and/or the mid infrared spectral range (MidIR), especially the light having a wavelength of 1 μm to 5 μm, preferably of 1 μm to 3 μm. This is due to the fact that many material properties or properties on the chemical constitution of many objects may be derived from the near infrared spectral range. It shall be noted, however, that spectroscopy in other spectral ranges is also feasible and within the scope of the present invention.

The term “light source” as used herein is a broad term and is to be given its ordinary and customary meaning to a person of ordinary skill in the art and is not to be limited to a special or customized meaning. The term specifically may refer, without limitation, to an arbitrary device configured for generating or providing light in the sense of the above-mentioned definition. The light source specifically may be or may comprise at least one electrical light source, such as an electrically driven light source. For example, the light source may comprise at least one light-emitting diode (LED).

In spectroscopy, various sources and paths of light are to be distinguished. In the context of the present invention, a nomenclature is used which, firstly, denotes light propagating from the light source to the object as “illumination light”. Secondly, light propagating from the object to the detector is denoted as “detection light”. The detection light may comprise at least one of illumination light reflected by the object, illumination light scattered by the object, illumination light transmitted by the object, luminescence light generated by the object, e.g. phosphorescence or fluorescence light generated by the object after optical, electrical or acoustic excitation of the object by the illumination light or the like. Thus, the detection light may directly or indirectly be generated through the illumination of the object by the illumination light.

The term “optical spectral range” as used herein is a broad term and is to be given its ordinary and customary meaning to a person of ordinary skill in the art and is not to be limited to a special or customized meaning. The term specifically may refer, without limitation, to a wavelength range comprising one or more of the infrared, the visible and the ultraviolet spectral range as defined above.

The illumination light has the optical spectral range at least partially located in the near-infrared spectral range. For example, the optical spectral range of the illumination light may comprise a wavelength range from 760 nm to 5 μm, specifically a wavelength range from 1 μm to 3 μm.

The spectrometer device comprises at least one sample interface configured for allowing the emitted light of the light source to illuminate an object held by the sample interface and configured for allowing light from the object held by the sample interface to propagate via the optical filter to the detector. The term “sample interface” as used herein is a broad term and is to be given its ordinary and customary meaning to a person of ordinary skill in the art and is not to be limited to a special or customized meaning. The term specifically may refer, without limitation, to a port of the spectrometer device through which light in the optical spectral range, such as in at least one partition of the optical spectral range or in the full optical spectral range, may enter the spectrometer device, specifically for the purpose of the spectral sensing, and/or may leave the spectrometer device, e.g. for the purpose of illuminating the at least one object. The sample interface, as an example, may define an optical plane, e.g. a plane either material or imaginary, of the spectrometer device, through which the illumination light from the first optical path, as will be explained in further detail below, may travel to reach the object and/or through which the detection light from the object may travel to reach the detector, e.g. to generate a detector signal. The sample interface may be a fictional plane. The sample interface may or may not be constituted by a physical element and/or barrier, such as a transparent element, e.g. a glass or quartz window. The sample interface may also be the sample surface itself or a plane where the sample can be placed or aligned. The sample interface may be arranged in an optical path of the spectrometer device, specifically in the first optical path, to allow the light emitted from the light source to illuminate the object placed in front of the spectrometer device, specifically in front of the sample interface. The term “passing the sample interface” as used herein is a broad term and is to be given its ordinary and customary meaning to a person of ordinary skill in the art and is not to be limited to a special or customized meaning. The term specifically may refer, without limitation, to one or more of propagating to the sample interface and from the sample interface, traversing the sample interface, and interacting with the sample interface. The emitted light may impinge on the sample interface, e.g. under an angle of incidence. The emitted light may interact with the sample interface and may leave the sample interface, e.g. under an exit angle. A point or region of impingement on the sample interface may be at the same side of the sample interface or on an opposing side as the point or region of exit from the sample interface.

The sample interface comprises at least one module cover window. The term “module” as used herein is a broad term and is to be given its ordinary and customary meaning to a person of ordinary skill in the art and is not to be limited to a special or customized meaning. The term specifically may refer, without limitation, to a complex element, in this case within the sample interface, that provides at least one function. The term “cover” as used herein is a broad term and is to be given its ordinary and customary meaning to a person of ordinary skill in the art and is not to be limited to a special or customized meaning. The term specifically may refer, without limitation, to an element configured for at least partially enclosing at least one other element. The term “window” as used herein is a broad term and is to be given its ordinary and customary meaning to a person of ordinary skill in the art and is not to be limited to a special or customized meaning. The term specifically may refer, without limitation, to an optically transparent optical element having a defined index of refraction and thickness. The window may provide an interface to an exterior of the spectrometer device, e.g. to an exterior of a housing of the spectrometer device. The term “module cover window” as used herein is a broad term and is to be given its ordinary and customary meaning to a person of ordinary skill in the art and is not to be limited to a special or customized meaning. The term specifically may refer, without limitation, to a module configured for providing at least one covering function and providing at least one window.

The module cover window comprises at least one substrate. The term “substrate” as used herein is a broad term and is to be given its ordinary and customary meaning to a person of ordinary skill in the art and is not to be limited to a special or customized meaning. The term specifically may refer, without limitation, to a base element on which further elements of the sample interface such as by coating can be applied.

The substrate may be at least partially transparent at least one given wavelength range. Specifically, the substrate may be comprise at least one semi-transparent material. The term “semi-transparent” as used herein is a broad term and is to be given its ordinary and customary meaning to a person of ordinary skill in the art and is not to be limited to a special or customized meaning. The term specifically may refer, without limitation, to a property of an optical element of partially transmitting incident light within at least one wavelength range. Specifically, a semi-transparent optical element may be configured for transmitting a part of incident light, whereas the other part of the incident light may be reflected at the semi-transparent optical element. The material of the substrate may specifically be semi-transparent at least in a selected wavelength range. The wavelength range for which the substrate is semi-transparent may be selected such that it matches at least partially the wavelength range of the light source. However, the wavelength range for which the substrate may be different from the wavelength range of the light source, e.g. narrower. Thus, the substrate at least partially may function as window. The substrate may be configured for allowing light of at least one wavelength range to pass through following Snell's law. For example, the substrate may be a glass substrate, e.g. a transparent glass window. For example, the glass substrate may comprise at least one material selected from the group consisting of: fused silica, soda lime, borosilicate, sapphire, Silicon, Germanium, Calcium fluoride, Magnesium fluoride, Barium fluoride, and the like. A top and/or a bottom surface may comprise an anti-reflective coating (AR-coating), e.g. to enhance the total transmission in the wavelengths range of interest and for an angle of incidence (AOI).

The substrate may be is at least partially structured, e.g. through sand blasting. For example, the substrate may be at least partially structured in an area comprising the internal calibration target. The structure may allow ensuring a desired reflection profile, e.g. Lambertian.

The module cover window comprises at least one internal calibration target partially covering the substrate.

The term “calibration target” as used herein is a broad term and is to be given its ordinary and customary meaning to a person of ordinary skill in the art and is not to be limited to a special or customized meaning. The term specifically may refer, without limitation, to a reference object having a known, such as a predetermined and/or a predefined, interaction with light. The calibration target can be used to calibrate the spectrometer device.

The term “internal calibration target” as used herein is a broad term and is to be given its ordinary and customary meaning to a person of ordinary skill in the art and is not to be limited to a special or customized meaning. The term specifically may refer, without limitation, to at least one calibration target integrated in the spectrometer device, in particular in the second optical path of the spectrometer device. Specifically, the internal calibration target may be configured for interacting with the illumination light in the second optical path in a predetermined or predefined manner. The internal calibration target may be configured for receiving the illumination light emitted from the light source and transferring the illumination light via the second optical path onto the optical filter. In particular, the internal calibration target may be configured for ensuring that the illumination light emitted from the light source propagates via the second optical path to the optical filter without passing the sample interface, e.g. by interacting with the illumination light, such as by reflecting and/or scattering and/or filtering the illumination light onto the optical filter.

The internal calibration target may comprise at least one diffusive reflective surface. The term “diffuse reflective surface” as used herein is a broad term and is to be given its ordinary and customary meaning to a person of ordinary skill in the art and is not to be limited to a special or customized meaning. The term specifically may refer, without limitation, to a surface configured for scattering incident light at a plurality of different angles, e.g. in ideal case Lambertian reflection. The internal calibration target may have diffusive scattering properties and a reflectance not equal to zero. Specifically, the internal calibration target may function as a diffuse mirror. The internal calibration target may comprise a surface roughness configured for generating a Lambertian reflection profile. The surface roughness may be selected such that a specular reflection on the surface has a Lambertian profile due to a ratio of the wavelength of the incident radiation and the surface roughness, thereby creating a “diffuse mirror”. The surface roughness may be selected depending on the wavelength of the impinging light, e.g. such that the reflection (e.g. Fresnel reflection generated by a metal or partial Fresnel and partial diffuse reflection generated by a dielectric material) has a Lambertian profile.

The term “interacting with the internal calibration target” as used herein is a broad term and is to be given its ordinary and customary meaning to a person of ordinary skill in the art and is not to be limited to a special or customized meaning. The term specifically may refer, without limitation, to at least one process of one or more of reflecting, absorbing, or transmitting.

For example, the internal calibration target comprises one or more of: at least one layer of Polytetrafluoroethylene (PTFE), at least one optical coating, such as a white surface coating, e.g. available from Nextel® Suede coating 3101, a dielectric coating, at least one partially reflective dielectric mirror, at least one metal coating or metal foil comprising one or more of gold, silver, aluminum, chromium, and at least one beam splitter. For example, the internal calibration target may be applied to the substrate by one or more of spray coating, stamping, printing, gluing, at least one squeegee process.

The substrate is at least partially covered by the internal calibration target. The internal calibration target may cover 0.1 to 99.9% of the substrate, specifically 0.5 to 25% of the substrate, more specifically 1 to 10% of the substrate. For example, 1 to 10% of the substrate may be covered by the internal calibration target and a rest, e.g. of 99 to 90% may be used for the first optical path for measuring the object.

The internal calibration target may comprise at least one white reflection element such as a coating, film or foil. The term “white” as used herein is a broad term and is to be given its ordinary and customary meaning to a person of ordinary skill in the art and is not to be limited to a special or customized meaning. The term specifically may characterize, without limitation, the reflection of the element, i.e. that the reflection is essentially wavelength independent in the wavelength range of interest, in particular that there are no dominant absorption peaks in the reflection spectrum of the element in the given wavelength range. The term “white reflection element” as used herein is a broad term and is to be given its ordinary and customary meaning to a person of ordinary skill in the art and is not to be limited to a special or customized meaning.

The term specifically may refer, without limitation, to a material layer having an at least partially diffusive reflective surface. The white reflection element may provide a diffusive reflective surface. The white reflection element may be semi-transparent at at least one given wavelength range. The wavelength range for which the white reflection element is semi-transparent may be selected such that it matches at least partially the wavelength range of the light source. However, the wavelength range for which the white reflection element may be different from the wavelength range of the light source, e.g. narrower. The white reflection element may be configured for at least partially reflecting and/or partially transmitting incident light. The white reflection element may comprise one or more of at least one layer of Polytetrafluoroethylene (PTFE), at least one optical coating, such as a white surface coating, e.g. a white varnish available from Nextel® Suede coating 3101.

The white reflection element may be arranged directly on the substrate. The term “arranged directly” as used herein is a broad term and is to be given its ordinary and customary meaning to a person of ordinary skill in the art and is not to be limited to a special or customized meaning. The term specifically may refer, without limitation, to application without any further element between the substrate and the white reflection element. For example, white reflection element may be coated, e.g. spray coated, on the substrate. However, other processes may be possible such as stamping, printing, gluing, at least one squeegee process and the like.

The internal calibration target may comprise at least one carrier element via which the white reflection element is arranged onto the substrate. The term “carrier element” as used herein is a broad term and is to be given its ordinary and customary meaning to a person of ordinary skill in the art and is not to be limited to a special or customized meaning. The term specifically may refer, without limitation, to at least one element and/or layer arranged between two other elements, e.g. the substrate and the white reflection element. The carrier element may be configured for providing at least one supporting function for mounting at least one further element of the internal calibration target onto the substrate. For example, the white reflection element and the carrier element may function together as internal calibration target and/or the carrier element may support the white reflection element. The carrier element may be diffuse reflective in at least one wavelength range. The carrier element may comprise at least one reflection material. The white reflection element may be semi-transparent. The carrier element may be configured for scattering light upon transmission of the white reflection element. Thus, the reflective carrier can increase overall reflectivity. The mounting of the white reflection element onto the carrier element may be performed e.g. using a transparent adhesive, e.g., epoxy glue, or in at least one spray coating (varnish) process. However, other processes are possible.

In one embodiment, the module cover window may comprise a stack. The stack may comprise at least the white reflection element, the carrier element and the substrate. In one embodiment, the stack may comprise the carrier element, with the white reflection element coated thereon, wherein the carrier element is coated directly onto the substrate. For example, the carrier element comprises at least one metal layer comprising one or more of gold, silver, aluminum, chromium, wherein the metal layer is coated onto the substrate, e.g. in at least one thin-film process.

In an embodiment, the stack may comprise the carrier element, with the white reflection element coated thereon, which is mounted to the substrate by using at least one adhesive. For example, the carrier element comprises a metal film fixed to the substrate by means of an adhesive. In one embodiment, the stack may comprise the carrier element mounted to the substrate by using at least one adhesive and the white reflection element is mounted to the carrier element by using at least one transparent adhesive. For example, the carrier element comprises a metal film which is fixed by an adhesive on the substrate, wherein the white reflective element is a reflective coating which is fixed by a transparent adhesive on the carrier element.

For example, the white reflective element comprises at least one layer of PTFE and the carrier element further may comprise at least one aluminum foil functioning as a reflector. The layer of PTFE may be mounted on the aluminum foil using a transparent adhesive. The PTFE may function as diffusor and can in combination with the reflector function as internal calibration target.

The module cover window is at least partially structured. The term “structured” as used herein is a broad term and is to be given its ordinary and customary meaning to a person of ordinary skill in the art and is not to be limited to a special or customized meaning. The term specifically may refer, without limitation, to a non-uniform distribution of one element.

For example, the internal calibration target may be non-uniformly distributed on the substrate. For example, the internal calibration target may comprise at least one pattern. The term “pattern” as used herein is a broad term and is to be given its ordinary and customary meaning to a person of ordinary skill in the art and is not to be limited to a special or customized meaning. The term specifically may refer, without limitation, to an arbitrary structured element. The pattern may be at least one pattern selected from the group consisting of a dot pattern, a checkerboard pattern or a random pattern, or any other pattern, which feature a macroscopic 2D homogeneity over the surface area of the calibration target. The pattern may be embodied such that the pattern only partially covers the substrate. For example, the white reflection element may be structured in such a way that itself is scattering the light for a given wavelength range.

As outlined above, for example, the substrate may be is at least partially structured, e.g. through sand blasting. The substrate may be structured partially or completely, e.g. such that it may have the desired reflection profile (e.g. Lambertian). The substrate may be partially coated with a metallic layer, which also serves as the blocking layer.

The term “optical path” as used herein is a broad term and is to be given its ordinary and customary meaning to a person of ordinary skill in the art and is not to be limited to a special or customized meaning. The term specifically may refer, without limitation, to a trajectory of light in the spectrometer device. The optical path of light in the spectrometer device may be affected by reflection, refraction, dispersion and/or absorption at one or more optical elements, such as lenses, prisms, mirrors, gratings or the like, comprised by the spectrometer device. The terms “first” and “second”, as generally used herein, are used for nomenclature, only, without implying any ranking or numbering.

The term “first optical path” as used herein is a broad term and is to be given its ordinary and customary meaning to a person of ordinary skill in the art and is not to be limited to a special or customized meaning. The term specifically may refer, without limitation, to an optical path with interaction of the illumination light at the object. Specifically, a detector signal obtained via the first optical path may be affected from a presence and/or an absence of the object at the spectrometer device, specifically at the sample interface of the spectrometer device. For example, a detector signal obtained via the first optical path having the object applied to the spectrometer device may be different from a detector signal obtained via the first optical path having no object applied to the spectrometer device, specifically irrespective of constant environmental conditions. In particular, the first optical path may be configured for allowing the illumination light emitted from the light source to propagate via the optical filter to the detector by passing the sample interface at least once. Specifically, the first optical path may allow the illumination light emitted from the light source to propagate to the sample interface and, subsequently, via the optical filter to the detector. Via the first optical path, the illumination light emitted from the light source may be guided directly or indirectly, such as by reflection, refraction and/or dispersion, to the sample interface. The first optical path may be partially arranged outside the spectrometer device, such as outside a housing of the spectrometer device. Specifically, the illumination light in the first optical path may leave the spectrometer device, in particular a housing of the spectrometer device, at the sample interface to illuminate the object arranged outside the spectrometer device. The first optical path may be configured for coupling the detection light reflected at the object back into the spectrometer device. The detection light may be guided from the sample interface directly or indirectly, such as by reflection, refraction and/or dispersion, to the optical filter and subsequently to the detector. The reflection at the sample interface may comprise a diffuse reflection. Specifically, the detection light in the first optical path illuminating the optical filter and subsequently the detector may be light diffusively reflected at the object. The detector may be configured for generating at least one detector signal in response to an illumination by incident light via the first optical path.

The term “second optical path” as used herein is a broad term and is to be given its ordinary and customary meaning to a person of ordinary skill in the art and is not to be limited to a special or customized meaning. The term specifically may refer, without limitation, to an optical path without interaction of the illumination light at the object. Specifically, a detector signal obtained via the second optical path may be unaffected from a presence and/or an absence of the object at the spectrometer device. For example, a detector signal obtained via the second optical path having the object applied to the spectrometer device, specifically to the sample interface, may be equal to a detector signal obtained via the second optical path having no object applied to the spectrometer device, specifically assuming constant environmental conditions. In particular, the second optical path may be configured for allowing the illumination light emitted from the light source to propagate via the optical filter to the detector without passing the sample interface, specifically without being reflected at the object. Via the second optical path, the illumination light emitted from the light source may be passed to the internal calibration target and subsequently to the optical filter and to the detector without interacting with the object. The second optical path may be arranged completely in the spectrometer device, such as within a housing of the spectrometer device. The illumination light following the second optical path may be emitted by the light source and may be directly or indirectly, such as by reflection, refraction and/or dispersion, guided to the internal calibration target and subsequently to the optical filter and the detector. As an example, the second optical path may comprise a fiber coupled optical path transferring light from the light source to the internal calibration target reflecting the illumination light to the optical filter. Alternatively or additionally, the second optical path may be configured for direct illumination of the internal calibration target with the illumination light emitted from the light source. The detector may be configured for generating at least one detector signal in response to an illumination by incident light via the second optical path.

The module cover window may comprise at least one stray light suppression element configured for suppressing of unwanted light hitting the object and/or for suppressing of stray light. The substrate may be at least partially covered by the stray light suppression element. The term “stray light” as used herein is a broad term and is to be given its ordinary and customary meaning to a person of ordinary skill in the art and is not to be limited to a special or customized meaning. The term specifically may refer, without limitation, to light originating from at least one source different from the object causing a signal in addition to the measurement signal dedicated for the selected wavelength ranges. For example, the stray light may be generated by one or more of interference, reflections, scattering at at least one element of the spectrometer device, e.g. an optical grating, the housing and the like. The term “stray light suppression element” as used herein is a broad term and is to be given its ordinary and customary meaning to a person of ordinary skill in the art and is not to be limited to a special or customized meaning. The term specifically may refer, without limitation, to at least one element configured for reducing stray light. The stray light suppression element may be black material. The stray light suppression element may have a reflectance and a transmittance about zero. The stray light suppression element may cover a fraction of the substrate, e.g. at the region where the internal calibration target is arranged. The stray light suppression element may be part of the module cover window. The stray light suppression element may comprises a coating, wherein the coating is made of black varnish e.g. Nextel® Black velvet. The stray light suppression element may be applied to the substrate by one or more of spray coating, stamping, printing, at least one squeegee process. For example, the stray light suppression element is arranged directly on the substrate. For example, the stray light suppression element is arranged via at least one further carrier element onto the substrate. For example, the further carrier element may comprise at least one element selected from the group consisting of: at least one metal foil such as an aluminum foil, e.g. Acktar; at least one plastic foil; at least one plastic film. The further carrier element may be mounted onto the substrate using at least one adhesive material. Using of a further carrier element can be advantageous since a shape of the coating can be pre-processed and/or no masks or others are required.

The white reflection element may be arranged adjacent to the stray light suppression element. For example, the white reflection element and the stray light suppression element are arranged side by side on the substrate. For example, both of the white reflection element and the stray light suppression element may be coatings on the substrate which were arranged next to each other.

For example, the white reflection element and the stray light suppression element are arranged in the stack of the module cover window. The white reflection element may be arranged to at least partially covering the stray light suppression element. For example, the stack may comprise the white reflection element applied onto the stray light suppression element, optionally mounted on the further carrier element, which is mounted on the substrate. For example, the white reflection element is mounted onto the stray light suppression element via at least one transparent adhesive. Alternatively, the stray light suppression element may be or may comprise at least one intransparent adhesive. The white reflection element may be glued via the intransparent adhesive on the (further) carrier element. A partial absorption of light in the adhesive layer can be prevented. This can allow preventing the absorption spectrum of the adhesive to be seen in the reflection spectrum of the white reflective material.

The spectrometer device may comprises at least one evaluation unit configured for evaluating at least one detector signal generated by the detector. The term “evaluation unit” as used herein is a broad term and is to be given its ordinary and customary meaning to a person of ordinary skill in the art and is not to be limited to a special or customized meaning. The term specifically may refer, without limitation, to an arbitrary device or a combination of devices configured to evaluate or process at least one first item of information, in order to generate at least one second item of information thereof. Thus, specifically, the evaluation unit may be configured for processing at least one input signal and to generate at least one output signal thereof. The at least one input signal, as an example, may comprise at least one detector signal provided directly or indirectly by the at least one detector. As an example, the evaluation unit may be or may comprise one or more integrated circuits, such as one or more application-specific integrated circuits (ASICs), and/or one or more data processing devices, such as one or more of computers, digital signal processors (DSP), field programmable gate arrays (FPGA) preferably one or more microcomputers and/or microcontrollers. Additional components may be comprised, such as one or more preprocessing devices and/or data acquisition devices, such as one or more devices for receiving and/or preprocessing of the detector signals, such as one or more AD-converters and/or one or more filters. Further, the evaluation unit may comprise one or more data storage devices. Further, the evaluation unit may comprise one or more interfaces, such as one or more wireless interfaces and/or one or more wire-bound interfaces.

The at least one evaluation unit may be adapted to execute at least one computer program, such as at least one computer program performing or supporting the step of generating the items of information. As an example, one or more algorithms may be implemented which, by using the at least one detector signal, may perform a predetermined transformation for determining the spectroscopic information on the object, such as for determining at least one spectroscopic information describing at least one property of the object. For this purpose, the evaluation unit may, particularly, comprise at least one data processing device, also referred to as a processor, in particular an electronic data processing device, which can be designed to generate the desired information by evaluating the detector signal. The evaluation unit may use an arbitrary process for generating the required information, such as by calculation and/or using at least one stored and/or known relationship. The evaluation unit specifically may be configured for performing at least one digital signal processing (DSP) technique on the primary detector signal or any secondary detector signal derived thereof, in particular at least one Fourier transformation. Additionally or alternatively, the evaluation unit may be configured for performing one or more further digital signal processing techniques on the primary detector signal or any secondary detector signal derived thereof, e.g. windowing, filtering, Goertzel algorithm, cross-correlation and autocorrelation. Besides the detector signal, one or a plurality of further parameters and/or items of information can influence said relationship. The relationship can be determined or determinable empirically, analytically or else semi-empirically. As an example, the relationship may comprise at least one of a model or calibration curve, at least one set of calibration curves, at least one function or a combination of the possibilities mentioned. One or a plurality of calibration curves can be stored for example in the form of a set of values and the associated function values thereof, for example in a data storage device and/or a table. Alternatively or additionally, however, the at least one calibration curve can also be stored for example in parameterized form and/or as a functional equation. Separate relationships for processing the detector signals into the items of information may be used. Alternatively, at least one combined relationship for processing the detector signals is feasible. Various possibilities are conceivable and can also be combined.

The spectrometer device may be configured for illuminating the detector with light having at least one wavelength λᵢ via the at least one first optical path to obtain at least one first detector signal S_{1,i}. The object may comprise at least one external calibration target. The external calibration target may be applied by a user to the sample interface of the spectrometer device. The spectrometer device may be configured for illuminating the detector with light having the at least one wavelength λᵢ via the at least one second optical path to obtain at least one calibration signal S_{2,i}. The evaluation unit may be configured for determining at least one item of calibration information cᵢ by using the first detector signal S_{1,i} and the calibration signal S_{2,i}. The determining of the item of calibration information may also be referred to as “initial calibration”. Specifically, the item of calibration information may be determined according to

$${c}_{i} = \frac{{S}_{1 , i}}{{S}_{2 , i}} .$$

The term “item of calibration information” as used herein is a broad term and is to be given its ordinary and customary meaning to a person of ordinary skill in the art and is not to be limited to a special or customized meaning. The term specifically may refer, without limitation, to an arbitrary item of information suitable for calibrating the spectrometer device. For example, the item of calibration information may comprise information on one or more of a wavelength calibration, a dark current calibration and an intensity calibration. The item of calibration information may be used for correcting, adjusting and/or compensating measurement signals at the spectrometer device. The item of calibration information may specifically comprise information on a relation of a measurement signal of the spectrometer device to a known calibration standard, specifically to the external calibration target.

The item of calibration information may be determined in an initial calibration of the spectrometer device, for example in a factory calibration. Additionally, it may be possible to re-determine the item of calibration information in-field, such as at a user site, by re-performing the steps of the initial calibration using the external calibration target. The item of calibration information may be stored, such as in a data storage device of the spectrometer device and/or in a data storage device accessible to the spectrometer device, and may be used for determining the spectroscopic information of the object using the spectrometer device. Specifically, the item of calibration information may be applied to a measurement signal of the spectrometer device, specifically in order to correct, adjust and/or compensate the measurement signal, to obtain a calibrated measurement signal, such as a measurement signal being calibrated to a known calibration standard, specifically to the external calibration target. The calibrated measurement signal may directly provide the spectroscopic information on the object or, alternatively, may be used to derive the spectroscopic information on the object.

The spectrometer device may be configured for illuminating the detector with light having at least one wavelength λᵢ; via the at least one first optical path to obtain at least one measurement signal S_{meas,i}. In this example, the object may comprise at least one measurement object, e.g. at least one object to be investigated using the spectrometer device. The spectrometer device may further be configured for illuminating the detector with light having the at least one wavelength λᵢ; via the at least one second optical path to obtain at least one reference signal S_{ref,i}. The evaluation unit may be configured for determining the spectroscopic information on the object using the item of calibration information cᵢ, the reference signal S_{ref,i} and the first detector signal. For example, the spectroscopic information of the object may be a reflectance R_{object,i}, wherein

$${R}_{object , i} = \frac{{S}_{meas , i}}{{c}_{i} * {S}_{ref , i}} .$$

In a calibration of the spectrometer device, for example in a factory calibration, the object applied to the spectrometer device, for example by assistance of a user, may comprise the external calibration target. A reflectance R_{λ,ext} of the external calibration target and a reflectance R_{λ,int} of the internal calibration target may be proportional to each other with

$${R}_{λ , ext} = a \cdot {R}_{λ , i n t} ,$$

wherein a is a constant.

The reflectance R_{λ,ext} of the external calibration target and the reflectance R_{λ,int} of the internal calibration target may be proportional to each other for each wavelength in the selected wavelength range. The constant a may be independent of temperature and/or wavelength.

The external calibration target may comprise a standardized diffuse reflectance target having a reflectance >99% over a range from 400 to 1500 nm and >95% from 250 to 2500 nm. For example, the external calibration target may comprise a commercially available calibration target, such as a Spectralon® Diffuse Reflectance Target. These reflectance targets may comprise thermally and/or chemically stable reflectance panels. These reflectance targets may comprise plates up to 24×24 inch of white or gray material, mounted in a rugged anodized aluminum frame. The reflectance of these reflectance targets may generally be >99% over a wavelength range from 400 to 1500 nm and >95% over a wavelength range from 250 to 2500 nm. Spectralon® Diffuse Reflectance Standards are available in plates up to 24×24 inch in the following reflectance values: 99%, 80%, 60%, 40%, 20%, 10%, 5% and 2% (10″ target max).

The internal calibration target may specifically be designed for emulating the reflectance of the external calibration target. For example, the external calibration target may have a first material and the internal calibration target may have a second material. The first material and the second material may be matched to each other such that the reflectance R_{λ,ext} of the external calibration target and the reflectance R_{λ,int} of the internal calibration target are proportional to each other.

In a further aspect of the present invention, a method of calibrating a spectrometer device is disclosed. The term “calibrating, also referred to as “calibration”, as used herein is a broad term and is to be given its ordinary and customary meaning to a person of ordinary skill in the art and is not to be limited to a special or customized meaning. The term specifically may refer, without limitation, to a process of correcting, adjusting and/or compensating measurement signals at the spectrometer device. The calibration process may comprise determining at least one item of calibration information, which may comprise at least one item of information on a result of the calibration process, such as a calibration function, a calibration factor, a calibration matrix or the like, and may be used for transforming one or more measured values into one or more calibrated or “true” values. The calibration of the spectrometer device may comprise at least one of a wavelength calibration, a dark current calibration and an intensity calibration. The calibration may comprise at least one two-step process, wherein, in a first step, information on a relation of a measurement signal of the spectrometer device to a known calibration standard, specifically to the external calibration target, is determined, wherein, in a second step, this information is used for correcting and/or adjusting the measurement signal of the spectrometer device, e.g. in order to reduce, minimize and/or eliminate deviations of the measurement signal from the known calibration standard. Thus, the calibration may comprise applying the item of calibration information, for example to a measurement signal and/or to a measurement spectrum of the spectrometer device. A calibration of the spectrometer device may improve and/or maintain accuracy of measurements performed with the calibrated spectrometer device.

In the method, the spectrometer device to be calibrated is a spectrometer device according to the present invention, such as according to any one of the embodiments disclosed above and/or according to any one of the embodiments disclosed in further detail below. Thus, for possible embodiments and definitions related to the spectrometer device, reference is made to the description of the spectrometer device.

The method comprises the following steps that may be performed in the given order. However, a different order may also be possible. In particular, one, more than one or even all of the method steps may be performed once or repeatedly. Further, the method steps may be performed successively or, alternatively, one or more of the method steps may be performed in a timely overlapping fashion or even in a parallel fashion and/or in a combined fashion. The method may further comprise additional method steps that are not listed.

The method comprises:
I. providing the object to the sample interface of the spectrometer device, wherein the object comprises at least one external calibration target;
II. illuminating the detector with light having at least one wavelength λᵢ via the at least one first optical path to obtain at least one first detector signal S_{1,i};
III. illuminating the detector with light having the at least one wavelength λᵢ; via the at least one second optical path to obtain at least one calibration signal S_{2,i}; and
IV. determining at least one item of calibration information cᵢ by using the first detector signal S_{1,i} and the calibration signal S_{2,i}.

Specifically, the item of calibration information may be determined according to

$${c}_{i} = \frac{{S}_{1 , i}}{{S}_{2 , i}} .$$

The method may specifically comprise repeating steps II. to IV. for a plurality of wavelengths i.

In a further aspect of the present invention, a method of determining at least one spectroscopic information of at least one object by using a spectrometer device is disclosed. In the method, the spectrometer device to be used is a spectrometer device according to the present invention, such as according to any one of the embodiments disclosed above and/or according to any one of the embodiments disclosed in further detail below. Thus, for possible embodiments and definitions related to the spectrometer device, reference is made to the description of the spectrometer device.

The method comprises the following steps that may be performed in the given order. However, a different order may also be possible. In particular, one, more than one or even all of the method steps may be performed once or repeatedly. Further, the method steps may be performed successively or, alternatively, one or more of the method steps may be performed in a timely overlapping fashion or even in a parallel fashion and/or in a combined fashion. The method may further comprise additional method steps that are not listed.

The method comprises:
i. providing the object to the sample interface, wherein the object comprises at least one measurement object;
ii. illuminating the detector with light having at least one wavelength λᵢ via the first optical path to obtain at least one measurement signal S_{meas,i};
iii. illuminating the detector with light having at least one wavelength A via the second optical path to obtain at least one reference signal S_{ref,i}; and
iv. determining the spectroscopic information of the object by using the measurement signal S_{meas,i}, the reference signal S_{ref,i} and the item of calibration information cᵢ determined by using the method of calibrating a spectrometer device according to the present invention, such as according to any one of the embodiments disclosed above and/or according to any one of the embodiments disclosed in further detail below.

The spectroscopic information of the object may specifically one or more of reflectance, transmittance, absorbance of the object. For example, the spectroscopic information of the object may be a reflectance R_{object,i}, wherein

$${R}_{object , i} = \frac{{S}_{meas , i}}{{c}_{i} * {S}_{ref , i}} .$$

The method may comprise repeating steps ii. to iv. for a plurality of wavelengths i.

In a further aspect of the present invention, a computer program is disclosed, comprising instructions which, when the program is executed by a computer or computer network, cause the computer or computer network to perform the method of calibrating a spectrometer device according to the present invention, such as according to any one of the embodiments disclosed above and/or according to any one of the embodiments disclosed in further detail below, and/or the method of determining at least one spectroscopic information of at least one object according to the present invention, such as according to any one of the embodiments disclosed above and/or according to any one of the embodiments disclosed in further detail below.

In a further aspect of the present invention, a computer-readable storage medium is disclosed, specifically a non-transient computer-readable storage medium, comprising instructions which, when the instructions are executed by a computer or computer network, cause the computer or computer network to perform the method of calibrating a spectrometer device according to the present invention, such as according to any one of the embodiments disclosed above and/or according to any one of the embodiments disclosed in further detail below, and/or the method of determining at least one spectroscopic information of at least one object according to the present invention, such as according to any one of the embodiments disclosed above and/or according to any one of the embodiments disclosed in further detail below.

As used herein, the term “computer-readable storage medium” specifically may refer to nontransitory data storage means, such as a hardware storage medium having stored thereon computer-executable instructions. The storage medium specifically may be or may comprise a storage medium such as a random-access memory (RAM) and/or a read-only memory (ROM).

The spectrometer device and the methods according to the present invention, in one or more of the above-mentioned embodiments and/or in one or more of the embodiments described in further detail below, provide a large number of advantages over known devices and methods of similar kind. Specifically, the spectrometer device having the transmission profiles of the optical filter of the first and the second optical paths proportional to each other may provide an internal calibration path which may specifically be designed such that the temperature-induced drift of the second optical path is identical to the temperature-induced drift of the first optical path having the object applied to the spectrometer device. Thus, the spectrometer device may provide a decreased temperature dependence.

The spectrometer device and the methods according to the present invention may specifically provide an optical design for the internal calibration path for spectrometer devices that show a dependence of the spectrum, e.g. on the x-axis and/or on the y-axis, on a direction from where light is collected, e.g. from the angle-of-incidence, for example for spectrometer devices having a dispersive element with an angle dependence. In one example, the dispersive element may be an optical filter, specifically an interferometric filter, such as a set of narrow bandpass filter. The spectrometer device and the methods according to the present invention may specifically apply to spectrometer device using open port light and/or a reference emitter for calibration of the spectrometer device.

The spectrometer device according to the present invention may specifically comprise matching transmission profiles of the optical filter for the first optical path and the second optical path, wherein T_{1,NBP}=d·T_{2,NBP} and d being a constant which is independent of temperature, wavelengths and/or angles. The transmission profile of the optical filter may depend on a distribution of angles of light hitting the optical filter. Thus, for matching the transmission profiles in the first optical path and in the second optical path, the spectrometer device according to the present invention may provide a matched angle distribution of light in the first optical path and in the second optical path on the optical filter. The matching transmission profiles of the optical filter for both optical paths may provide an improved temperature compensation of the light source. Additionally, in case the reflectance of the internal calibration target is matched to the reflectance of the external calibration target, such that R_{λ,ext}=a*R_{λ,int} for all wavelength in the wavelength region of interest, the temperature compensation of the light source can be even more improved, specifically such that the item of calibration information may be constant for all temperatures.

The spectrometer device according to the present invention may specifically provide the same effective transmission profile for light in the first optical path and in the second optical path. The transmission profile of the optical filter may depend on a distribution of incident light on the optical filter. In one embodiment, the internal calibration target may be arranged in a particular manner to match the transmission profiles which may specifically provide an improved open-port calibration scheme for the spectrometer device. In one embodiment, the spectrometer device may comprise a reference light source arranged symmetrically to the light source with respect to the optical filter, which may specifically provide an improved calibration scheme using a second reference light source. The constant d may specifically yield a scaling of the item of calibration information cᵢ, but may not yield any unwanted temperature dependence.

In a further aspect according to the present invention, a use of a spectrometer device according to the present invention is disclosed, in an application selected from the group consisting of: an infrared detection application; a spectroscopy application; an exhaust gas monitoring application; a combustion process monitoring application; a pollution monitoring application; an industrial process monitoring application; a mixing or blending process monitoring; a chemical process monitoring application; a food processing process monitoring application; a food preparation process monitoring; a water quality monitoring application; an air quality monitoring application; a quality control application; a temperature control application; a motion control application; an exhaust control application; a gas sensing application; a gas analytics application; a motion sensing application; a chemical sensing application; a mobile application; a medical application; a mobile spectroscopy application; a food analysis application; an agricultural application, in particular characterization of soil, silage, feed, crop or produce, monitoring plant health; a plastics identification and/or recycling application; a healthcare and/or beauty application, in particular a determining of skin hydration and/or oxygen saturation, e.g. in an animal or human skin, a determining of an oxygen saturation in blood, urine, saliva or other bodily fluids, a non-invasive blood sugar measurement or tracking application, a long term blood sugar measurement or tracking application, a body temperature measurement or tracking application.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically are used only once when introducing the respective feature or element. In most cases, when referring to the respective feature or element, the expressions “at least one” or “one or more” are not repeated, nonwithstanding the fact that the respective feature or element may be present once or more than once.

Further, as used herein, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Summarizing and without excluding further possible embodiments, the following embodiments may be envisaged:

Summarizing and without excluding further possible embodiments, the following embodiments may be envisaged:
Embodiment 1: A spectrometer device for obtaining spectroscopic information on at least one object, the spectrometer device comprising
   at least one detector for detecting light from an object;
   at least one optical filter configured for transferring incident light within at least one selected wavelength range onto the detector;
   at least one light source configured for emitting light in at least one optical spectral range;
   at least one sample interface configured for allowing the emitted light of the light source to illuminate an object held by the sample interface and configured for allowing light from the object held by the sample interface to propagate via the optical filter to the detector, wherein the sample interface comprises at least one module cover window comprising at least one substrate, wherein the module cover window comprises at least one internal calibration target partially covering the substrate;
   at least one first optical path, wherein the first optical path is configured for allowing the emitted light of the light source to propagate via the optical filter to the detector by passing the sample interface at least once; and
   at least one second optical path, wherein the second optical path is configured for allowing the emitted light of the light source to propagate via the optical filter to the detector by interacting with the internal calibration target at least once.
Embodiment 2: The spectrometer device according to the preceding embodiment, wherein the internal calibration target comprises at least one diffusive reflective surface.
Embodiment 3: The spectrometer device according to any one of the preceding embodiments, wherein the internal calibration target functions as a diffuse mirror, wherein the internal calibration target comprises a surface roughness configured for generating a Lambertian reflection profile
Embodiment 4: The spectrometer device according to any one of the preceding embodiments, wherein the internal calibration target comprises one or more of: at least one layer of Polytetrafluoroethylene (PTFE), at least one optical coating, such as a white surface coating, a dielectric coating, at least one partially reflective dielectric mirror, at least one metal coating or metal foil comprising one or more of gold, silver, aluminum, chromium, and at least one beam splitter.
Embodiment 5: The spectrometer device according to any one of the preceding embodiments, wherein the internal calibration target comprises at least one white reflection element, or at least one white reflection element and at least one carrier element.
Embodiment 6: The spectrometer device according to the preceding embodiment, wherein the carrier element is diffuse reflective in at least one wavelength range, wherein the white reflection element is semi-transparent at at least one given wavelength range.
Embodiment 7: The spectrometer device according to any one of the preceding embodiments, wherein the module cover window is at least partially structured, wherein the internal calibration target comprises at least one pattern, wherein the pattern is arranged such that the pattern partially covers the substrate, wherein the pattern is at least one pattern selected from the group consisting of a dot pattern, a checkerboard pattern or a random pattern, or any other pattern, which feature a macroscopic 2D homogeneity over the surface area of the calibration target.
Embodiment 8: The spectrometer device according to any one of the preceding embodiments, wherein the substrate is a glass substrate.
Embodiment 9: The spectrometer device according to any one of the preceding embodiments, wherein the substrate is at least partially covered by the internal calibration target, wherein the internal calibration target covers 0.1 to 99.9% of the substrate, specifically 0.5 to 25% of the substrate, more specifically 1 to 10% of the substrate.
Embodiment 10: The spectrometer device according to any one of the preceding embodiments, wherein the module cover window comprises at least one stray light suppression element configured for suppressing of unwanted light hitting the object and/or for suppressing of stray light.
Embodiment 11: The spectrometer device according to the preceding embodiment, wherein the stray light suppression element has a reflectance and a transmittance about zero.
Embodiment 12: The spectrometer device according to any one of the two preceding embodiments, wherein the stray light suppression element covers a fraction of the substrate.
Embodiment 13: The spectrometer device according to any one of the three preceding embodiments, wherein the stray light suppression element is arranged directly on the substrate or the stray light suppression element is arranged via at least one further carrier element onto the substrate.
Embodiment 14: The spectrometer device according to any one of the four preceding embodiments, wherein the stray light suppression element comprises a coating, wherein the coating is made of black varnish.
Embodiment 15: The spectrometer device according to any one of the five preceding embodiments, wherein the stray light suppression element is applied to the substrate by one or more of spray coating, stamping, printing, at least one squeegee process.
Embodiment 16: The spectrometer device according to any one of the six preceding embodiments, wherein the stray light suppression element is arranged via a further carrier element onto the substrate, wherein the further carrier element comprises at least one element selected from the group consisting of: at least one metal foil such as aluminum; at least one plastic foil; at least one plastic film.
Embodiment 17: The spectrometer device according to the preceding embodiment, wherein the further carrier element is mounted onto the substrate using at least one adhesive material.
Embodiment 18: The spectrometer device according to any one of the preceding embodiments, wherein the spectrometer device is a near-infrared spectrometer.
Embodiment 19: The spectrometer device according to any one of the preceding embodiments, wherein the emitted light has the optical spectral range at least partially located in the near-infrared spectral range.
Embodiment 20: The spectrometer device according to any one of the preceding embodiments, wherein the spectrometer device comprises at least one evaluation unit configured for evaluating at least one detector signal generated by the detector.
Embodiment 21: A method of calibrating a spectrometer device, wherein the spectrometer device is a spectrometer device according to any one of the preceding embodiments, the method comprising:
   I. providing the object to the sample interface of the spectrometer device, wherein the object comprises at least one external calibration target;
   II. illuminating the detector with light having at least one wavelength λᵢ; via the at least one first optical path to obtain at least one first detector signal S_{1,i};
   III. illuminating the detector with light having the at least one wavelength λᵢ via the at least one second optical path to obtain at least one calibration signal S_{2,i}; and
   IV. determining at least one item of calibration information cᵢ by using the first detector signal S_{1,i} and the calibration signal S_{2,i}.
Embodiment 22: A method of determining at least one spectroscopic information of at least one object by using a spectrometer device according to any one of the preceding embodiments referring to a spectrometer device, the method comprising:
   i. providing the object to the sample interface, wherein the object comprises at least one measurement object;
   ii. illuminating the detector with light having at least one wavelength λᵢ via the first optical path to obtain at least one measurement signal S_{meas,i};
   iii. illuminating the detector with light having at least one wavelength λᵢ via the second optical path to obtain at least one reference signal S_{ref,i}; and
   iv. determining the spectroscopic information of the object by using the measurement signal S_{meas,i}, the reference signal S_{ref,i} and the item of calibration information cᵢ determined by using the method of calibrating a spectrometer device according to the preceding claim.
Embodiment 23: A use of a spectrometer device according to any one of the preceding embodiments referring to a spectrometer device, in an application selected from the group consisting of: an infrared detection application; a spectroscopy application; an exhaust gas monitoring application; a combustion process monitoring application; a pollution monitoring application; an industrial process monitoring application; a mixing or blending process monitoring; a chemical process monitoring application; a food processing process monitoring application; a food preparation process monitoring; a water quality monitoring application; an air quality monitoring application; a quality control application; a temperature control application; a motion control application; an exhaust control application; a gas sensing application; a gas analytics application; a motion sensing application; a chemical sensing application; a mobile application; a medical application; a mobile spectroscopy application; a food analysis application; an agricultural application, in particular characterization of soil, silage, feed, crop or produce, monitoring plant health; a plastics identification and/or recycling application; a healthcare and/or beauty application, in particular a determining of skin hydration and/or oxygen saturation, e.g. in an animal or human skin, a determining of an oxygen saturation in blood, urine, saliva or other bodily fluids, a non-invasive blood sugar measurement or tracking application, a long term blood sugar measurement or tracking application, a body temperature measurement or tracking application.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an embodiment of a spectrometer device for obtaining spectroscopic information on at least one object in a schematic view; and

FIGS. 2 to 5C show different embodiments of the sample interface of the spectrometer device in schematic views;

FIG. 6 shows a flow chart of an embodiment of a method of calibrating a spectrometer device; and

FIG. 7 shows a flow chart of an embodiment of method.

### DETAILED DESCRIPTION OF THE EMBODIMENTS

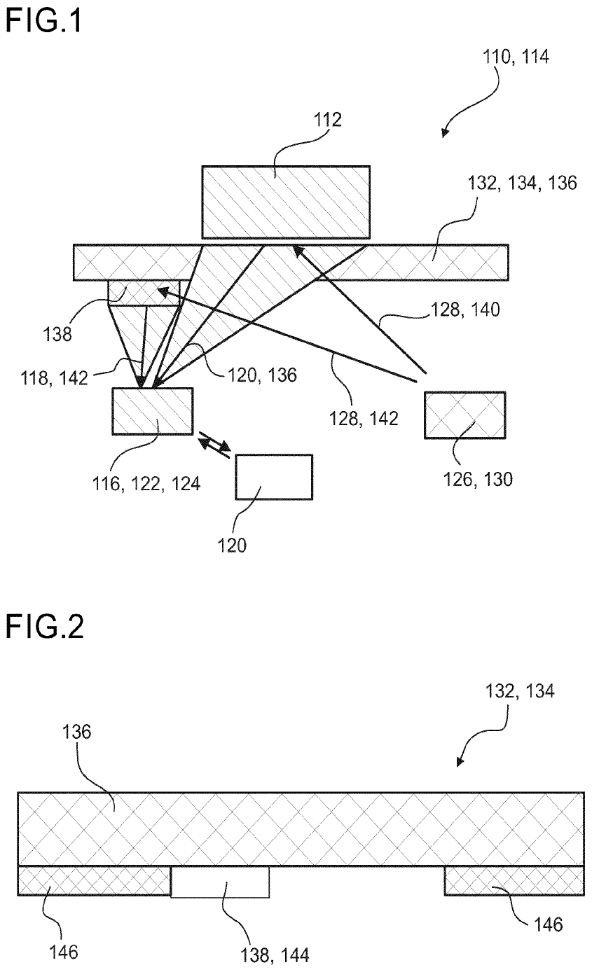

FIG. 1 shows an exemplary embodiment of a spectrometer device 110 for obtaining spectroscopic information on at least one object 112 in a schematic view. The spectrometer device 110 may specifically be a near-infrared spectrometer 114.

The spectrometer device 110 comprises at least one detector 116 for detecting light, in particular detection light 118 from the object 112. The detector 116 may comprise one single optically sensitive element or area or a plurality of optically sensitive elements or areas (not shown in the Figures). Specifically, the detector 116 may be or may comprise at least one detector array, more specifically an array of photosensitive elements. Each of the photosensitive elements may comprise at least a photosensitive area which may be adapted for generating an electrical signal depending on the intensity of the incident light, wherein the electrical signal may, in particular, be provided to an evaluation unit 120. The spectrometer device 110 may specifically comprise at least one evaluation unit 120 for evaluating at least one detector signal generated by the detector 116 and for determining the spectroscopic information on the object 112 using the detector signal.

The spectrometer device 110 further comprises at least one optical filter 122 configured for transferring incident light within at least one selected wavelength range onto the detector 116. A transmission profile of the optical filter 122 may be dependent on an angle-of-incidence. As can be seen in FIG. 1, the optical filter 122 may be arranged adjacent to the detector 116, such as by placing the optical filter 122 on top of the detector 116. The optical filter 122, as an example, may comprise at least one narrow band pass filter 124, more specifically a set of narrow band pass filter 124. The narrow band pass filter 124 may be configured for transmitting light only within a narrow selected wavelength range, such as a wavelength range having a width in the range of 10 to 100 nm, specifically in the range of 10 to 50 nm, more specifically a wavelength range having a width of 20 nm, most specifically a wavelength range having a width of 15 nm. For example, in the set of narrow band pass filter 124, each of the narrow band pass filter 124 may have a narrow selected wavelength rang at least partially differing from each other.

The spectrometer device 110 further comprises at least one light source 126 configured for emitting light, specifically illumination light 128, in at least one optical spectral range. For example, the light source 126 may comprise at least one light-emitting diode (LED) 130. In the example of FIG. 1, the illumination light 128 may have the optical spectral range at least partially located in the near-infrared spectral range.

The spectrometer device 110 further comprises at least one sample interface 132 configured for allowing the emitted light of the light source 126 to illuminate an object 112 held by the sample interface 132 and configured for allowing light from the object 112 held by the sample interface 132 to propagate via the optical filter 122 to the detector 116. The sample interface 132 comprises at least one module cover window 134 comprising at least one substrate 136, wherein the module cover window 134 comprises at least one internal calibration target 138 partially covering the substrate 136. Different embodiments of the sample interface 132 and the module cover window 134 are shown in FIGS. 2 to 6. Thus, for a detailed description of the sample interface 132 and the module cover window 134, reference is made to the description of FIGS. 2 to 6.

The spectrometer device 110 further comprises at least one first optical path 140 and at least one second optical path 142. The first optical path 140 is configured for allowing the emitted light of the light source 126, specifically the illumination light 128, to propagate via the optical filter 122 to the detector 116 by passing the sample interface 132 at least once. The second optical path 142 is configured for allowing the emitted light of the light source 126, specifically the illumination light 128, to propagate via the optical filter 122 to the detector 116 by interacting with the internal calibration target 138 at least once.

FIGS. 2 to 6 show different exemplary embodiments of the sample interface 132 of the spectrometer device 110 in schematic views. In these exemplary embodiments shown in FIGS. 2 to 6, the substrate 136 may be a glass substrate

In the example of FIG. 2, the internal calibration target 138 may comprise at least one white reflection element 144 such as a coating, film or foil. The white reflection element 144 may provide a diffusive reflective surface. As shown in FIG. 2, the white reflection element 144 may be arranged directly on the substrate 136. Additionally, the module cover window 134 may comprise at least one stray light suppression element 146 configured for suppressing of unwanted light hitting the object 112 and/or for suppressing of stray light. The stray light suppression element 146 may be black material. The stray light suppression element 146 may have a reflectance and a transmittance about zero. The stray light suppression element 146 may cover a fraction of the substrate 136, e.g. at the region where the internal calibration target 138 is arranged. The stray light suppression element 146 may be part of the module cover window 134.

The substrate 136 may be at least partially covered by the stray light suppression element 146. The stray light suppression element 146 may comprises a coating, wherein the coating is made of black varnish e.g. Nextel® Black velvet. The stray light suppression element 146 may be applied to the substrate 136 by one or more of spray coating, stamping, printing, at least one squeegee process. For example, as can be seen in FIG. 2, the stray light suppression element 146 may be arranged directly on the substrate 136. In the exemplary embodiment of FIG. 2, the white reflection element 144 may be arranged adjacent to the stray light suppression element 146. For example, the white reflection element 144 and the stray light suppression element 146 may be arranged side by side on the substrate 136. For example, both of the white reflection element 144 and the stray light suppression element 146 may be coatings on the substrate 136 which were arranged next to each other. However, other alternative arrangements are also feasible.

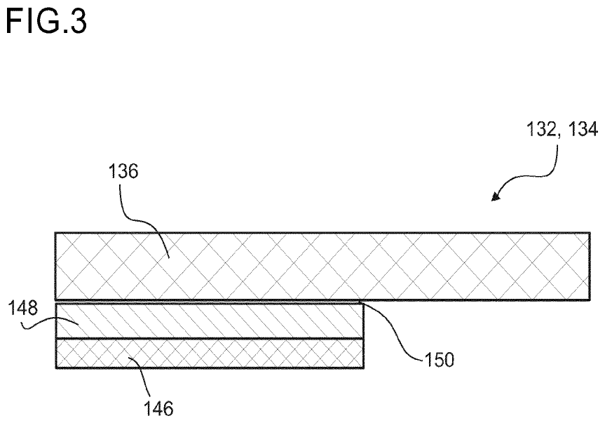

FIG. 3 shows an exemplary embodiment of the sample interface 132 similar to the embodiment of FIG. 2, wherein, in this example, the stray light suppression element 146 is arranged via at least one further carrier element 148 onto the substrate 136; in this case without any internal calibration target. For example, the further carrier element 148 may comprise at least one element selected from the group consisting of: at least one metal foil such as an aluminum foil, e.g. Acktar; at least one plastic foil; at least one plastic film. The further carrier element 148 may be mounted onto the substrate 136 using at least one adhesive material 150. Using of a further carrier element 148 can be advantageous since a shape of the coating can be pre-processed and/or no masks or others are required.

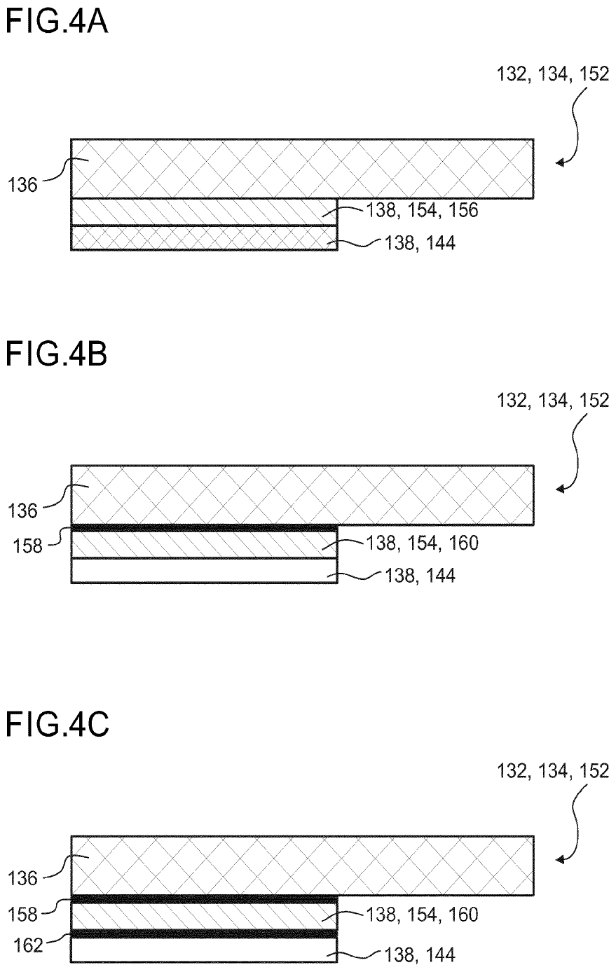

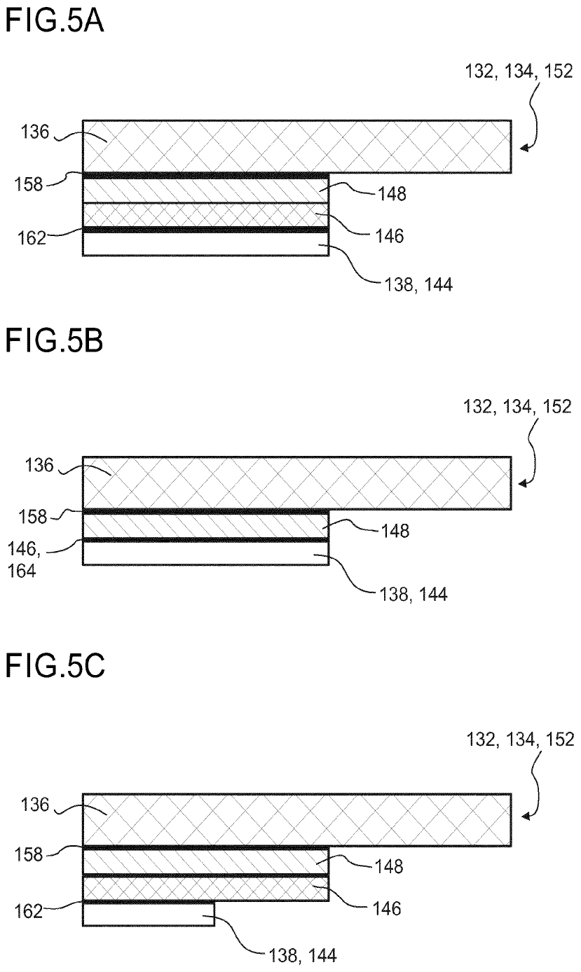

In the exemplary embodiments of FIGS. 4A to 5C, the module cover window 134 may comprise a stack 152. Additionally, the internal calibration target 138 may comprise at least one carrier element 154 via which the white reflection element 144 is arranged onto the substrate 136. The stack 152 may comprise at least the white reflection element 144, the carrier element 154 and the substrate 136.

For example, as shown in FIG. 4A, the stack 152 may comprise the carrier element 154 with the white reflection element 144 coated thereon, wherein the carrier element 154 is coated directly onto the substrate 136. For example, the carrier element 154 comprises at least one metal layer 156 comprising one or more of gold, silver, aluminum, chromium, wherein the metal layer 156 is coated onto the substrate 136, e.g. in at least one thin-film process.

Additionally or alternatively, as shown in FIG. 4B, the stack 152 may comprise the carrier element 154 with the white reflection element 144 coated thereon. The carrier element 154 may be mounted to the substrate 136 by using at least one adhesive 158. For example, the carrier element 154 comprises a metal film 160 fixed to the substrate 136 by means of the adhesive 158.

Additionally or alternatively, as shown in FIG. 4C, the stack 152 may comprise the carrier element 154 mounted to the substrate 136 by using the at least one adhesive 158 and the white reflection element 144 is mounted to the carrier element 154 by using at least one transparent adhesive 162. For example, the carrier element 154 comprises the metal film 160 which is fixed by the adhesive 158 on the substrate 136, wherein the white reflective element 144 is a reflective coating which is fixed by the transparent adhesive 162 on the carrier element 154.

In the exemplary embodiments of FIGS. 5A to 5C, additionally or alternatively, the white reflection element 144 and the stray light suppression 146 element may be arranged in the stack 152 of the module cover window 134. The white reflection element 144 may be arranged to at least partially covering the stray light suppression element 146.

For example, as shown in FIG. 5A, the stack 152 may comprise the white reflection element 144 applied onto the stray light suppression element 146 mounted on the further carrier element 148, which is mounted on the substrate 136, e.g. via an adhesive 158. The white reflection element 144 may be mounted onto the stray light suppression element 146 via at least one transparent adhesive 162.

Alternatively, as shown in FIG. 5B, the stray light suppression element 146 may be or may comprise at least one intransparent adhesive 164. The white reflection element 144 may be glued via the intransparent adhesive 164 on the further carrier element 148. A partial absorption of light in the adhesive layer 158 can be prevented. This can allow preventing the absorption spectrum of the adhesive 158 to be seen in the reflection spectrum of the white reflective material.

For example, as shown in FIG. 5C, the stack 152 may be embodied similar to the stack 152 of FIG. 5A, wherein the white reflection element 144 may be arranged to partially cover the stray light suppression element 146.

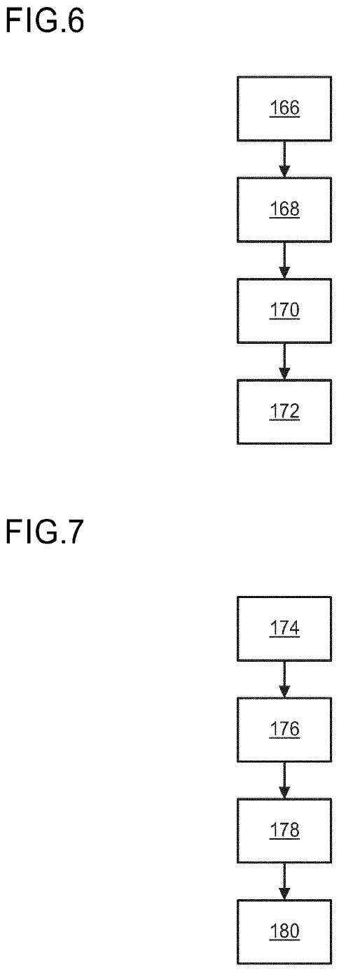

FIG. 6 shows a flow chart of an exemplary embodiment of a method of calibrating a spectrometer device 110. In the method, the spectrometer device 110 to be calibrated is a spectrometer device 110 according to the present invention, such as according to the embodiment shown in FIG. 1 and/or according to any other embodiment disclosed herein. Thus, for a description of the spectrometer device 110, reference is made to the description of FIG. 1.

The method comprises the following steps that may be performed in the given order. However, a different order may also be possible. In particular, one, more than one or even all of the method steps may be performed once or repeatedly. Further, the method steps may be performed successively or, alternatively, one or more of the method steps may be performed in a timely overlapping fashion or even in a parallel fashion and/or in a combined fashion. The method may further comprise additional method steps that are not listed.

The method comprises:
I. (denoted by reference number 166) providing the object 112 to the sample interface 132 of the spectrometer device 110, wherein the object 112 comprises at least one external calibration target;
II. (denoted by reference number 168) illuminating the detector 116 with light having at least one wavelength λᵢ via the at least one first optical path 140 to obtain at least one first detector signal S_{1,i};
III. (denoted by reference number 170) illuminating the detector 116 with light having the at least one wavelength λᵢ; via the at least one second optical path 142 to obtain at least one calibration signal S_{2,i}; and
IV. (denoted by reference number 172) determining at least one item of calibration information cᵢ by using the first detector signal S_{1,i} and the calibration signal S_{2,i}.

Specifically, the item of calibration information may be determined according to

$${c}_{i} = \frac{{S}_{1 , i}}{{S}_{2 , i}} .$$

The method may specifically comprise repeating steps II. to IV. for a plurality of wavelengths i.

FIG. 7 shows a flow chart of an exemplary embodiment of a method of determining at least one spectroscopic information of at least one object 112 by using a spectrometer device 110. In the method, the spectrometer device 110 to be used is a spectrometer device 110 according to the present invention, such as according to the embodiment shown in FIG. 1 and/or according to any other embodiment disclosed herein. Thus, for a description of the spectrometer device 110, reference is made to the description of FIG. 1.

The method comprises the following steps that may be performed in the given order. However, a different order may also be possible. In particular, one, more than one or even all of the method steps may be performed once or repeatedly. Further, the method steps may be performed successively or, alternatively, one or more of the method steps may be performed in a timely overlapping fashion or even in a parallel fashion and/or in a combined fashion. The method may further comprise additional method steps that are not listed.

The method comprises:
i. (denoted by reference number 174) providing the object 112 to the sample interface 132, wherein the object 112 comprises at least one measurement object;
ii. (denoted by reference number 176) illuminating the detector 116 with light having at least one wavelength λᵢ via the first optical path 140 to obtain at least one measurement signal S_{meas,i};
iii. (denoted by reference number 178) illuminating the detector 116 with light having at least one wavelength λᵢ via the second optical path 142 to obtain at least one reference signal S_{ref,i}; and
iv. (denoted by reference number 180) determining the spectroscopic information of the object 112 by using the measurement signal S_{meas,i}, the reference signal S_{ref,i} and the item of calibration information cᵢ determined by using the method of calibrating a spectrometer device 110 according to the present invention, such as according to the embodiment shown in FIG. 6 and/or according to any other embodiment disclosed herein.

The spectroscopic information of the object 112 may specifically one or more of reflectance, transmittance, absorbance of the object 112. For example, the spectroscopic information of the object 112 may be a reflectance R_{object,i}, wherein

$${R}_{object , i} = \frac{{S}_{meas , i}}{{c}_{i} * {S}_{ref , i}} .$$

The method may comprise repeating steps ii. to iv. for a plurality of wavelengths i.

### LIST OF REFERENCE NUMBERS

110 spectrometer device
112 object
114 near-infrared spectrometer
116 detector
118 detection light
120 evaluation unit
122 optical filter
124 narrow band pass filter
126 light source
128 illumination light
130 light-emitting diode
132 sample interface
134 module cover window
136 substrate
138 internal calibration target
140 first optical path
142 second optical path
144 white reflection element
146 stray light suppression element
148 further carrier element
150 adhesive material
152 stack
154 carrier element
156 metal layer
158 adhesive
160 metal film
162 transparent adhesive
164 intransparent adhesive
166 providing the external calibration target
168 illuminating the detector via the first optical path
170 illuminating the detector via the second optical
172 determining at least one item of calibration information
174 providing the measurement object
176 illuminating the detector via the first optical path
178 illuminating the detector via the second optical path
180 determining the spectroscopic information of the object

1. A spectrometer device for obtaining spectroscopic information on at least one object, the spectrometer device comprising
at least one detector for detecting light from an object;
at least one optical filter configured for transferring incident light within
at least one selected wavelength range onto the detector;
at least one light source configured for emitting light in at least one optical spectral range;
at least one sample interface configured for allowing the emitted light of the light source to illuminate an object held by the sample interface and configured for allowing light from the object held by the sample interface to propagate via the optical filter to the detector, wherein the sample interface comprises at least one module cover window comprising at least one substrate, wherein the module cover window comprises at least one internal calibration target partially covering the substrate, wherein the module cover window comprises at least one stray light suppression element configured for suppressing of unwanted light hitting the object and/or for suppressing of stray light, wherein the stray light suppression element has a reflectance and a transmittance about zero, wherein the stray light suppression element covers a fraction of the substrate;
at least one first optical path, wherein the first optical path is configured for allowing the emitted light of the light source to propagate via the optical filter to the detector by passing the sample interface at least once; and at least one second optical path, wherein the second optical path is configured for allowing the emitted light of the light source to propagate via the optical filter to the detector by interacting with the internal calibration target at least once.

2. The spectrometer device according to claim 1, wherein the internal calibration target comprises at least one diffusive reflective surface.

3. The spectrometer device according to claim 1, wherein the internal calibration target functions as a diffuse mirror, wherein the internal calibration target comprises a surface roughness configured for generating a Lambertian reflection profile.

4. The spectrometer device according to claim 1, wherein the internal calibration target comprises one or more of: at least one layer of Polytetrafluoroethylene, at least one optical coating, at least one white surface coating, at least one dielectric coating, at least one partially reflective dielectric mirror, at least one metal coating or metal foil comprising one or more of gold, silver, aluminum, chromium, and at least one beam splitter.

5. The spectrometer device according to claim 1, wherein the internal calibration target comprises at least one white reflection element, or at least one white reflection element and at least one carrier element, wherein the carrier element is diffuse reflective in at least one wavelength range, wherein the white reflection element is semi-transparent at at least one given wavelength range.

6. The spectrometer device according to claim 1, wherein the module cover window is at least partially structured, wherein the internal calibration target comprises at least one pattern, wherein the pattern is arranged such that the pattern partially covers the substrate, and wherein the pattern is at least one pattern selected from the group consisting of a dot pattern, a checkerboard pattern or a random pattern, and any other patterns that feature a macroscopic 2D homogeneity over the surface area of the calibration target.

7. The spectrometer device according to claim 1, wherein the substrate is a glass substrate.

8. The spectrometer device according to claim 1, wherein the substrate is at least partially covered by the internal calibration target,
wherein the internal calibration target covers 0.1 to 99.9% of the substrate.

9. The spectrometer device according to claim 1, wherein the stray light suppression element is arranged directly on the substrate or the stray light suppression element is arranged via at least one further carrier element onto the substrate.

10. The spectrometer device according to claim 1, wherein the stray light suppression element comprises a coating, wherein the coating is made of black varnish, and/or wherein the stray light suppression element is applied to the substrate by one or more of spray coating, stamping, printing, and at least one squeegee process.

11. The spectrometer device according to claim 1, wherein the stray light suppression element is arranged via a further carrier element onto the substrate, wherein the further carrier element comprises at least one element selected from the group consisting of at least one metal foil, at least one aluminum foil; at least one plastic foil; and at least one plastic film, wherein the further carrier element is mounted onto the substrate using at least one adhesive material.

12. A method of calibrating the spectrometer device according to claim 1, the method comprising:
I. providing the object to the sample interface of the spectrometer device, wherein the object comprises at least one external calibration target;
II. illuminating the detector with light having at least one wavelength Li via the at least one first optical path to obtain at least one first detector signal S_{1,i};
III. illuminating the detector with light having the at least one wavelength λᵢ via the at least one second optical path to obtain at least one calibration signal S_{2,i}; and
IV. determining at least one item of calibration information cᵢ by using the first detector signal S_{1,i} and the calibration signal S_{2,i}.

13. A method of determining at least one spectroscopic information of at least one object by using the spectrometer device according to claim 1, the method comprising:
i. providing the object to the sample interface, wherein the object comprises at least one measurement object;
ii. illuminating the detector with light having at least one wavelength λᵢ via the first optical path to obtain at least one measurement signal S_{meas,i};
iii. illuminating the detector with light having at least one wavelength λᵢ via the second optical path to obtain at least one reference signal S_{ref,i}; and
iv. determining the spectroscopic information of the object by using the measurement signal S_{meas,i}, the reference signal S_{ref,i} and the item of calibration information cᵢ determined by using a method of calibrating the spectrometer device comprising:
I. providing the object to the sample interface of the spectrometer device, wherein the object comprises at least one external calibration target;
II. illuminating the detector with light having at least one wavelength λᵢ via the at least one first optical path to obtain at least one first detector signal S_{1,i};
III. illuminating the detector with light having the at least one wavelength λᵢ via the at least one second optical path to obtain at least one calibration signal S_{2,i}; and
IV. determining at least one item of calibration information cᵢ by using the first detector signal S_{1,i} and the calibration signal S_{2,i}.

14. A method of using the spectrometer device according to any one of the preceding claims referring to a spectrometer device claim 1, the method comprising using the spectrometer device in an application selected from the group consisting of an infrared detection application; a spectroscopy application; an exhaust gas monitoring application; a combustion process monitoring application; a pollution monitoring application; an industrial process monitoring application; a mixing or blending process monitoring; a chemical process monitoring application; a food processing process monitoring application; a food preparation process monitoring; a water quality monitoring application; an air quality monitoring application; a quality control application; a temperature control application; a motion control application; an exhaust control application; a gas sensing application; a gas analytics application; a motion sensing application; a chemical sensing application; a mobile application; a medical application; a mobile spectroscopy application; a food analysis application; an agricultural application; a plastics identification and/or recycling application; and a healthcare and/or beauty application, a determining of skin hydration and/or oxygen saturation, a determining of an oxygen saturation in blood, urine, saliva or other bodily fluids, a non-invasive blood sugar measurement or tracking application, a long term blood sugar measurement or tracking application, and a body temperature measurement or tracking application.

15. The method according to claim 14, wherein the agricultural application comprises characterization of soil, silage, feed, crop or produce, or monitoring plant health.

16. The method according to claim 14, wherein the determining of skin hydration and/or oxygen saturation is in an animal or human skin.
